(19) United States
(12) Patent Application Publication
(10) Pub. No.: US 2025/0282060 A1
(43) Pub. Date: Sep. 11, 2025

(54) HANDLING SYSTEM FOR STERILE GOODS CONTAINERS AND METHOD FOR MACHINE HANDLING OF STERILE GOODS CONTAINERS

(71) Applicant: Aesculap AG, Tuttlingen (DE)

(72) Inventors: Arne Jansen-Troy, Stockach (DE); Allan Maas, Konstanz (DE); Daniel Kiessling, Villingen-Schwenningen (DE); Matthias Henke, Villingen-Schwenningen (DE)

(21) Appl. No.: 19/220,537

(22) Filed: May 28, 2025

Related U.S. Application Data
(63) Continuation of application No. PCT/EP2023/083315, filed on Nov. 28, 2023.

(30) Foreign Application Priority Data
Nov. 29, 2022 (DE) 10 2022 131 591.6

(51) Int. Cl. B25J 15/00; B66C 1/66

(52) U.S. Cl. CPC B25J 15/0033; B25J 15/0047; B66C 1/663

(57) ABSTRACT

A handling system for sterile goods containers, in particular for automatically opening sterile goods containers, includes at least one tool apparatus for opening sterile goods containers. The at least one tool apparatus has at least one first tool element for being brought into engagement with a locking bracket mounted on a sterile goods container lid of a sterile goods container so as to be pivotable about a pivot axis, and in a storage position is oriented in parallel or substantially in parallel with a lid top side of the sterile goods container lid for locking the sterile goods container lid on a sterile goods container tray of the sterile goods container in a closing position. The first tool element has a lifting wedge for engaging under the locking bracket. The handling system can be used in a method for machine handling of sterile goods containers.

### CROSS-REFERENCE TO RELATED APPLICATIONS

This application is a continuation, under 35 U.S.C. § 365, of International Application No. PCT/EP2023/083315, filed on Nov. 28, 2023, and claims priority to German Application No. 10 2022 131 591.6, filed on Nov. 29, 2022. The contents of International Application No. PCT/EP2023/083315 and German Application No. 10 2022 131 591.6 are incorporated by reference herein in their entireties.

### FIELD

The present disclosure relates to a handling system for sterile goods containers, in particular for automatically opening sterile goods containers.

Furthermore, the present disclosure relates to a method for machine handling of sterile goods containers, said containers comprising a sterile goods container tray and a sterile goods container lid, wherein the sterile goods container lid comprises two locking brackets, which are held on the sterile goods container lid so as to be pivotable about a pivot axis, for locking the sterile goods container lid on the sterile goods container tray in a closing position.

### BACKGROUND

Sterile goods containers are known in a wide variety of embodiments. As an example, reference is made in this regard to DE 10 2020 103 131 A1.

Sterile goods containers serve, in particular, for the transport and storage of medical instruments and implants. The sterile goods containers typically comprise a sterile goods container tray and a sterile goods container lid that closes said tray in a closing position. However, the medical instruments and implants are typically not stored directly in these sterile goods containers, but rather in sieve baskets that are accommodated in these sterile goods containers.

In the closing position, the sterile goods containers are locked. A locking mechanism is described as an example in DE 10 2018 117 046 A1. In order to transfer the sterile goods container from the locked closing position into an unlocking position, a locking bracket of the locking mechanism must be actuated, namely pivoted. Furthermore, WO 2008/078 169 A2 describes a sterile goods container with a closure mechanism that can be operated with one hand.

Until now, after a use in a sterile environment, sterile goods containers of that kind are typically unpacked by hand on the so-called “unclean” side, i.e. as in a non-sterile environment. This means that one or more closures, i.e., in particular a locking mechanism mentioned above, must be opened on the sterile goods container lid. The sterile goods container lid can then be removed from the sterile goods container tray. In a next step, a sieve basket accommodated in the sterile goods container can be removed from the sterile goods container tray.

This manual procedure for unpacking sterile goods containers is time-consuming and also strenuous for the personnel responsible for this. Filled sterile goods containers can easily weigh more than 10 kg, such that handling them is a genuine physical strain if an operator has to handle such sterile goods containers all day.

### SUMMARY

It is therefore an object of the present disclosure to improve a handling system for sterile goods containers and a method of the kind described at the outset in such a way that the handling of sterile goods containers is made easier.

This object is achieved, in accordance with the present disclosure, in a handling system for sterile goods containers of the kind described at the outset in that the handling system for sterile goods containers comprises at least one tool apparatus for opening sterile goods containers, in that the at least one tool apparatus comprises at least one first tool element for being brought into engagement with a locking bracket that is mounted on a sterile goods container lid of a sterile goods container so as to be pivotable about a pivot axis and in a storage position is oriented in parallel or substantially in parallel with a lid top side of the sterile goods container lid for locking the sterile goods container lid on a sterile goods container tray comprised by the sterile goods container in a closing position in which the sterile goods container lid closes the sterile goods container tray, and in that the first tool element has a lifting wedge for engaging under the locking bracket.

Further developing a handling system for sterile goods containers in the proposed way simplifies and facilitates, in particular, the handling of sterile goods containers. The at least one tool apparatus that is proposed in accordance with the present disclosure makes it possible in a simple manner to engage with the lifting wedge under a free end or a transverse web of a locking bracket on a sterile goods container lid and to then pivot the locking bracket about the pivot axis, namely through a movement of the at least one tool apparatus relative to the sterile goods container lid and thus also relative to the locking bracket. In particular, during such a relative movement, the locking bracket can slide on the lifting wedge and thus be forced guided. The proposed tool apparatus makes it possible, in particular, for sterile goods containers to be opened and unlocked by machine. An operator thus no longer has to open the sterile goods containers by hand, which can be stiff and are locked with a predetermined locking force. To this end, an operator can guide the at least one tool apparatus, for example, by hand. Alternatively, the at least one tool apparatus may also be arranged or held on a manipulation apparatus, for example on a robot, in order to thus fully automatically pivot the locking bracket by bringing the at least one tool apparatus into engagement with the lifting wedge comprised thereby.

It is favorable if the lifting wedge has a lifting wedge abutment face and a lifting wedge sliding face, if the lifting wedge abutment face, during the intended use of the at least one tool apparatus, is oriented in parallel with the lid top side, and if the lifting wedge abutment face and the lifting wedge sliding face enclose a wedge angle. A configuration of the lifting wedge of that kind makes it possible, in particular, to orient said lifting wedge with the lifting wedge abutment face in parallel with the lid top side and to also move it relative to the lid top side in order to then engage with the lifting wedge under the locking bracket on the sterile goods container lid. Here, the lifting wedge sliding face can then come into contact with the locking bracket and pivot the same in a forced manner upon a movement of the at least one tool apparatus and the locking bracket relative to one another.

In order to make it possible to easily open the sterile goods containers, it is advantageous if the lifting lever sliding face is of planar or curved configuration. In particular, it may be of concavely or convexly curved configuration pointing away from the lifting wedge. In particular, a concave curvature of the lifting wedge sliding face enables a simple and secure engagement under a locking bracket on a sterile goods container lid.

The wedge angle preferably has a value in a range of about 10° to about 50°. With a lifting wedge that has such a wedge angle, it is possible to reliably engage under a locking bracket.

It is favorable if the lifting wedge abutment face and the lifting wedge sliding face intersect in a wedge edge, and if a lifting wedge sliding face length of the lifting wedge sliding face in parallel with the wedge edge is greater, in particular more than five times greater, than a lifting wedge sliding face width perpendicular to the wedge edge. Such an embodiment makes it possible, in particular, to form a narrow lifting wedge, which is ultimately required to slightly raise the locking bracket out of the storage position with a short movement path, i.e., with a relative movement between the at least one tool apparatus and the locking bracket. It is thus possible, in particular, to form the at least one tool apparatus as compactly as possible.

It is advantageous if the at least one tool element has a tool element base body and if the lifting wedge is arranged or formed distally on the tool element base body. Thus, in particular, the lifting wedge can be formed as small as possible, but the tool element base body large and thus also more stable in order to, in particular, pivot the locking bracket, as is described in more detail in the following, further out of the storage position, also against possible forces exerted by the locking mechanism. The at least one tool apparatus, in particular, can thus be formed to be sufficiently stable for the desired application. In particular, the lifting wedge may be arranged or formed distally in such a way that it projects from the tool element base body both to the front and also pointing away from a top side of the tool element base body. It can thus be ensured, in particular, that a contact between the at least one tool apparatus and the locking bracket always takes place with the lifting wedge first and then the tool element base body is able to come into contact with the locking bracket.

It is favorable if the tool element base body has a base body abutment face and a base body sliding face for lifting and opening the locking bracket, and if the base body abutment face and the base body sliding face enclose a base body wedge angle. A configuration of that kind enables, in particular, a wedge-shaped form of the tool element base body. For example, to open the locking bracket, the tool element base body can be moved with the base body abutment face in parallel with the lid top side of the sterile goods container lid.

In order to enable an optimal movement of the at least one tool apparatus along and in parallel with the lid top side, it is favorable if the base body abutment face extends in parallel or substantially in parallel with the lifting wedge abutment face. The base body abutment face and the lifting wedge abutment face are preferably spaced at a distance from one another. The lifting wedge can thus project from the tool element base body to the front and to the bottom relative to said base body.

It is advantageous if the base body abutment face defines an abutment face plane and if the lifting wedge projects beyond the abutment face plane. As already explained, when it comes into contact with the locking bracket, the at least one tool apparatus can thus first act with the lifting wedge on the locking bracket in order to lift it slightly out of the storage position.

Preferably, the base body wedge angle has a value that corresponds to or substantially corresponds to the wedge angle of the lifting wedge. This makes it possible, in particular, to continuously pivot the locking bracket relative to the sterile goods container lid when the at least one tool apparatus acts on the locking bracket.

It is advantageous if the tool element base body has a base body top side, which defines the base body sliding face, and if a latching recess that is recessed relative to the base body sliding face is formed on the base body top side. Such a latching recess can accommodate, in particular, part of the locking bracket, for example a transverse web thereof forming a handle region, in order to couple the locking bracket to the at least one tool apparatus in a defined manner. For example, said transverse web may latch into the latching recess in a handling position.

In order to be able to handle and manipulate the sterile goods container with the at least one tool apparatus, in particular, in the desired manner, the latching recess favorable has a retaining face pointing away from the lifting wedge and a latching face extending in parallel or substantially in parallel with the base body sliding face. Such a configuration makes it possible, in particular, to accommodate the described transverse web of the locking bracket and to bring it into abutment against both the retaining face and the latching face. The retaining face makes it possible to prevent, in particular, the locking bracket from slipping off the at least one tool apparatus in an undesired manner. The retaining face therefore forms a stop face for part of the locking bracket when it is accommodated in the latching recess.

Preferably, the retaining face extends transversely, in particular perpendicularly, to the base body sliding face and defines a respective intersection line with the base body sliding face and the latching face. Thus, depending on a depth of the latching recess, a defined and reliable stop face for part of the locking bracket can be formed by the retaining face.

It is advantageous if the latching face has a latching face width perpendicular to the retaining face and if the latching face width is greater than a web width of a transverse web of the locking bracket extending perpendicular to the pivot axis. Thus, through the selection of the size of the latching face width, it can be ensured, in particular, that a transverse web of the locking bracket on the sterile goods container lid is able to be accommodated in the latching recess over its entire web width. Thus, in particular, with the at least one tool apparatus, it is also possible to hold the locking bracket under pretension relative to the sterile goods container lid if necessary, without the locking bracket being able to slip off the at least one tool apparatus.

It is favorable if the tool element base body defines a base body width parallel to the wedge edge and if the latching face extends over the entire base body width. It can thus be achieved, in particular, that a transverse web of the locking bracket is able to abut completely and, as the case may be, in surface-to-surface contact against the latching face. The retaining face can thus also exert its effect on an entire length, thus over the base body width.

It may further be advantageous if the tool element base body has a base body bottom side pointing away from the base body top side and if the base body bottom side comprises a base body bottom side face extending parallel with the base body sliding face. This configuration makes it possible to form the tool element base body in a substantially cuboidal shape. It can thus be formed particularly compactly and with as little material as possible. In particular, a weight of the at least one tool apparatus can thus be minimized.

In order to be able to optimally handle sterile goods containers with locking brackets of the described type using the handling system for sterile goods containers, it is advantageous if the locking bracket has an engagement portion, if the engagement portion defines an engagement portion width parallel with the pivot axis, and if a lifting wedge sliding face length and a base body width of the tool element base body parallel with the wedge edge are smaller than the engagement portion width. This configuration makes it possible, in particular, to not only bring the first tool element into engagement with the locking bracket, but also to pass it through the engagement portion of the locking bracket. Thus, in particular, a transverse web can be brought into engagement with a latching recess, which may optionally be provided on the first tool element as described above.

In accordance with a further preferred embodiment, provision may be made that the at least one tool apparatus comprises a first interface device for directly or indirectly coupling to a mechanical handling apparatus in a force-locking and/or positive-locking manner for moving, in particular for displacing, rotating, and/or pivoting, the at least one tool apparatus. The first interface device makes it possible, in particular, to selectively couple the at least one tool apparatus to a handling apparatus and to also remove it therefrom as necessary. Alternatively, the first interface device may also serve to couple the at least one tool apparatus to a handle, such that a user is also able to use the tool apparatus to pivot the locking bracket on the sterile goods container lid in a manner that is ergonomic for the user in order to unlock the sterile goods container lid. The first interface device makes it possible, in particular, to use the first tool apparatus in connection with handling apparatuses that are also used for other purposes. In addition, different tool apparatuses can thus also be used with the handling apparatus, in particular in order to handle different types of sterile goods containers.

Preferably, the first interface device comprises at least one first interface element, which is coupleable in a force-locking and/or positive-locking manner to a corresponding second interface element that is arranged or formed on the handling apparatus or is associated therewith. The interface elements may be configured, in particular, in the form of projections and recesses corresponding to said projections. In particular, the interface elements may comprise a threaded bore, a through-bore, and a screw element in order to couple the first tool apparatus to the handling apparatus, namely by screwing. Alternatively, bayonet-like interface elements for forming a bayonet connection are also conceivable. But also temporary force-locking connections, such as are able to be established, for example, by means of permanent magnets or electromagnets, are usable for coupling the first and second interface elements.

For handling sterile goods containers, it is favorable if the handling apparatus comprises a robot. In particular, said robot may comprise a robot arm with a plurality of articulated portions, which are pivotable relative to one another and optionally also rotatable about their respective longitudinal axis. It is thus possible, in particular, to orient the at least one tool apparatus and position it in space in the desired manner with the robot, for example relative to a sterile goods container, in particular to its sterile goods container lid.

It is advantageous if the handling apparatus comprises an actuator device, if the actuator device is directly or indirectly coupled or coupleable to the robot on the one hand and to the at least one tool apparatus on the other hand, and if the actuator device is configured to move the at least one tool apparatus, in particular to displace, rotate, and/or pivot the same. In particular, the actuator device may be coupleable to a robot as specified. The actuator device can thus be positioned in space and oriented in the desired manner, in particular relative to the sterile goods container lid. The actuator device also enables an additional movement of the at least one tool apparatus, independent of a movement of the robot. Thus, for example, the robot can position the actuator device relative to the sterile goods container lid. A movement of the at least one tool apparatus can then also be brought about by the actuator device. In particular, the actuator device can displace, rotate, and/or pivot the at least one tool apparatus in order to, for example, move, in particular pivot, a locking bracket on the sterile goods container in the desired manner.

In order to be able to move the at least one tool apparatus in the desired manner, it is advantageous if the actuator device comprises a drive. Said drive may be configured, in particular, in the form of an electromotive drive and/or a pneumatic drive. In particular, displacement movements of the at least one tool apparatus can thus be performed with the actuator device in a simple manner. The drive may comprise, in particular, gear spindles and/or piston-cylinder arrangements in order to move the at least one tool apparatus.

The drive is preferably configured in the form of a linear drive and/or a pivot drive and/or a rotary drive. The at least one tool apparatus can thus be displaced, rotated, or pivoted with the actuator device in a simple manner.

The actuator device advantageously comprises a second interface device for directly or indirectly coupling to the robot in a force-locking and/or positive-locking manner. This configuration makes it possible, in particular, to separate the actuator device with one or more tool apparatuses coupled thereto from the robot or to reconnect it to said robot. In particular, the second interface device may be a defined, standardized interface device, which are known for coupling apparatuses of any kind to a robot.

In principle, it is conceivable to connect the at least one tool apparatus directly to the actuator device, for example with pistons or threaded rods of corresponding drives of the actuator device. It is favorable if the at least one tool apparatus and the actuator device are indirectly connected to one another by way of an adapter element. Thus, for example, a standardized actuator device can be coupled to the at least one tool apparatus so that an actuation of the locking bracket in the desired manner is possible. In particular, spatial or geometric requirements in the use of the handling system for sterile goods containers can thus be fulfilled, for example in order to handle different types of sterile goods containers with the same tool apparatuses.

The handling system for sterile goods containers favorably comprises two tool apparatuses, which, in particular, are held directly or indirectly on the actuator device with lifting wedges pointing away from one another or pointing toward one another. This configuration makes it possible, in particular, to simultaneously or successively handle two locking mechanisms arranged on face sides of the sterile goods container that point away from one another, in particular to simultaneously or successively actuate the respective locking brackets with the two tool apparatuses in order to unlock the sterile goods container lid and to optionally also lift it from the sterile goods container tray.

Advantageously, the actuator device is configured to move the at least one tool apparatus in a path-controlled and/or force-controlled manner. In this way, on the one hand a position of the at least one tool apparatus relative to the sterile goods container and on the other hand forces exerted by the at least one tool apparatus on the sterile goods container, in particular on the locking bracket on the sterile goods container, can be predetermined. A risk of damage to the sterile goods containers by the handling system for sterile goods containers can thus be minimized.

For a simple structure and compact configuration of the tool apparatus, it is favorable if the at least one tool apparatus is of symmetrical configuration relative to a midplane extending perpendicularly to the wedge edge. In particular, the at least one tool apparatus may be of mirror-symmetrical configuration relative to the midplane.

In accordance with a further preferred embodiment, provision may be made that the at least one tool apparatus comprises a support element, and that the at least one first tool element is arranged or formed projecting transversely, in particular perpendicularly, relative to a support element longitudinal axis defined by the support element. With such a support element, it is possible, in particular, to arrange and position the at least one first tool element in the desired manner. In addition, such a support element also enables a simple configuration and arrangement of the first interface device described above. The at least one first tool element may optionally be configured to be connectable to the support element in a force-locking and/or positive-locking manner. Alternatively, a one-piece, in particular monolithic, configuration of the at least one first tool element and the support element is also possible.

It is favorable if the at least one tool apparatus comprises at least one second tool element for being brought into positive-locking or substantially positive-locking engagement with a swivel bracket of a sieve basket that is insertable into a sterile goods container and/or with a swivel bracket holding element holding the swivel bracket on the sieve basket so as to be pivotable about a swivel bracket axis. For example, the swivel bracket holding element may be configured in the form of a recess for a free end, bent by 90°, of one of two legs extending in parallel or substantially in parallel with one another of a swivel bracket that as a whole has a U-shape. The swivel bracket holding element may be arranged, in particular, on an end wall of the sieve basket, namely on a side face of the end wall that points toward an inner space of the sieve basket. Optionally, a support may also be provided on the swivel bracket holding element, which predetermines an orientation of the swivel bracket in a rest position, i.e., when not external forces are being exerted on the swivel bracket except for gravitational force. In the rest position, the pivot bracket is preferably oriented in parallel or substantially in parallel with a base of the sieve basket. The configuration of the at least one second tool element for being brought into engagement with a swivel bracket holding element has the advantage, in particular, that the sieve basket is able to be directly accessed. If the swivel bracket holding element is immovably held on the sieve basket, the sieve basket can thus be reliably and securely manipulated in the desired manner by bringing the swivel bracket holding element and the at least one second tool element into engagement.

For a compact structure of the tool apparatus, it is favorable, in particular, if the at least one second tool element is arranged or formed on the support element. In particular, it may be arranged or formed projecting from the support element, for example, in parallel with the first tool element, i.e., in the same direction as it.

The at least one second tool element advantageously comprises a coupling receptacle for being brought into positive-locking engagement with a swivel bracket holding element of a sieve basket. The swivel bracket holding element can thus, in particular, be brought into engagement with the at least one second tool element in a simple manner. To achieve this, no complex mechanism with movable parts has to be provided on the at least one second tool element, which would enable a force-locking coupling. In particular, the gravitational force can thus be used to couple the swivel bracket holding element and the at least one second tool element.

It is favorable if the at least one second tool element has a tool element top side and if the coupling receptacle is arranged or formed on the tool element top side. Thus, for example, the swivel bracket holding element can dip or engage into the coupling receptacle from above in order to enable a positive-locking engagement between the swivel bracket holding element and the at least one second tool element.

It is advantageous if the at least one first tool element and the at least one second tool element are separated from one another by a cutout. Such a cutout makes it possible, in particular, to pass through an engagement portion on the locking bracket with the at least one first tool element, even if the at least one tool apparatus has at least one second tool element provided. Thus, for example, a leg of a substantially U-shaped or O-shaped locking bracket is able to engage into the cutout between the at least one first and the at least one second tool element when the at least one first tool element passes through the engagement portion on the locking bracket.

The at least one tool apparatus preferably comprises two second tool elements. This configuration makes it possible, in particular, with the at least one tool apparatus to simultaneously bring each of the two second tool elements into engagement with a respective swivel bracket holding element, which are associated with a common swivel bracket. If two tool apparatuses of this kind are provided, then the four swivel bracket holding elements of the two swivel brackets of the sieve basket can be brought into engagement with the two tool apparatuses in a defined manner. This tool apparatuses coupled to the sieve basket in this manner can then be used, in particular, through corresponding manipulation, i.e., movement thereof in space, for example with a handling apparatus, to remove the sieve basket from the sterile goods container, for example from the sterile goods container tray. In particular, this may take place fully automatically if the handling apparatus is referenced in a predefined coordinate system. This coordinate system may be predefined, for example, by the sterile goods container, for example a corresponding mechanical or electronic element, which predetermines a coordinate system in three-dimensional space in a defined manner.

It is advantageous if the coupling receptacles of the two second tool elements define a common coupling receptacle longitudinal axis and if the coupling receptacle longitudinal axis extends in parallel or substantially in parallel with the swivel bracket pivot axis when a swivel bracket holding element is accommodated in each coupling receptacle of the two second tool elements. As described, the at least one tool apparatus thus does not engage on the swivel bracket itself, but instead on a holder of the swivel bracket, namely the swivel bracket holding elements comprised by such a holder. If said swivel bracket holding elements are fixed directly to the sieve basket, namely in an immovable manner, then the sieve basket can be directly manipulated by bringing the at least one tool apparatus into engagement with the two swivel bracket holding elements as described. In particular, it is thus also possible to handle a sterile goods container on which the swivel bracket is deformed or damaged. For example, a coupling of the at least one tool apparatus to the sieve basket is possible independently of a shape of the swivel bracket. If the swivel bracket holding elements are arranged or formed on the sieve basket in a standardized form, then a wide variety of sieve baskets can be brought into engagement with the same tool apparatus in a simple and secure manner.

The at least one first tool element is favorably arranged between the two second tool elements. In particular, a symmetrical configuration of the tool apparatus can thus be achieved. In particular, with such a tool apparatus, which comprises the two second tool elements and a first tool element, a sterile goods container lid on the one hand and a sieve basket on the other hand are able to be handled by machine in the described manner.

A simple and compact structure of the at least one tool apparatus can be achieved, in particular, by the two second tool elements being arranged or formed symmetrically relative to the midplane.

It may further be favorable if a transverse stop is arranged or formed on each coupling receptacle for delimiting a movement of the at least one tool apparatus relative to a sterile goods container in a direction parallel with the swivel bracket pivot axis when the swivel bracket holding elements are accommodated in the coupling receptacles. Thus, by means of such a transverse stop, an undesired movement of the sieve basket in a direction parallel to the swivel bracket pivot axis relative to the at least one tool apparatus can be prevented.

In order to minimize a relative movement of the sieve basket and the at least one tool apparatus, in particular aside from unavoidable play, it is advantageous if the transverse stop delimits the coupling receptacle laterally in the direction toward the at least one first tool element.

It is favorable if the at least one second tool element has a tool element abutment face, if the tool element abutment face defines part of a tool element bottom side of the at least one second tool element, and if the tool element abutment face extends in parallel or substantially in parallel with the base body abutment face and/or with the lifting wedge abutment face. Such a tool element abutment face may serve, in particular, to orient the at least one second tool element in parallel with a lid top side of a sterile goods container lid.

The tool element abutment face preferably lies in the abutment face plane or defines the latter. This configuration can ensure, in particular, that only the lifting wedge projects beyond the abutment face plane. Thus, even if two second tool elements are provided, the handling of a locking bracket on a sterile goods container lid can be reliably ensured, even when two second tool elements are additionally provided with which an engagement with a sieve basket is able to be established.

It is advantageous if the at least one second tool element has a tool element sliding face for lifting a swivel bracket of the sieve basket and if the tool element abutment face and the tool element sliding face enclose a tool element wedge angle. The tool element sliding face makes it possible, in particular, for a swivel bracket to slide thereon, said swivel bracket being oriented, as described, in parallel with a base of the sieve basket in a resting or transport position, in order to thus enable a reliable and unimpeded engagement of the swivel bracket holding elements into the coupling receptacles.

The tool element wedge angle preferably corresponds to or substantially corresponds to the base body wedge angle. It can thus be made possible, in particular, that the tool element sliding face and the base body sliding face define a common sliding face plane. In this way, it can then be prevented that the swivel bracket is able to dip with a transverse web extending in parallel with the swivel bracket pivot axis into the latching recess.

As already explained, it is advantageous if the tool element sliding face and the base body sliding face define a common sliding face plane. Thus, in particular, a transverse web of the swivel bracket, said web extending in parallel with the swivel bracket pivot axis, can be prevented from dipping into the latching recess.

It is further favorable if the at least one second tool element has a tool element bottom side pointing away from the tool element top side and if the tool element bottom side comprises a tool element bottom side face extending in parallel with the tool element sliding face. This embodiment makes it possible, in particular, to configure the at least one second tool element to be substantially cuboidal, except for a free end. The coupling receptacle may be arranged or formed in the region of the free end. It can thus be achieved that the at least one tool apparatus has a minimal weight.

The at least one tool apparatus can be configured to be particularly stable if it is formed in one piece, in particular monolithically.

It is further favorable if the at least one tool apparatus is made of a metallic material. In particular, it may be an instrument steel that is sterilizable by superheated steam. The at least one tool apparatus can thus be optimally cleaned and can also be used for an application in a sterile environment, as the case may be.

The object stated at the outset is further achieved, in accordance with the present disclosure, in a method of the kind described at the outset in that a handling system for sterile goods containers described above is provided, and in that the at least one tool apparatus is brought into engagement with a locking bracket for pivoting the same from the closing position, in which the locking bracket is oriented in parallel or substantially in parallel with the lid top side of the sterile goods container, about the pivot axis into an unlocking position, in which the sterile goods container lid and the sterile goods container tray are unlocked.

The proposed method makes it possible, in particular, to unlock and open a sterile goods container fully automatically. To this end, for example, one of the advantageous embodiments of handling systems for sterile goods containers described above can be used. It is thus possible to open sterile goods containers in a manner that is sustainable, comfortable, and less physically strenuous for an operator.

It is favorable if a transverse web of the locking bracket is accommodated in the latching recess on the at least one first tool element and is held therein under tension against the retaining face. In particular, in this way, the sterile goods container lid can be prevented from slipping off the at least one tool apparatus of the handling system for sterile goods containers. If, for example, two tool apparatuses are provided, which are each in engagement with a respective locking bracket in the described manner, the sterile goods container lid can thus be grasped with the two tool apparatuses and handled accordingly, for example lifted from a sterile goods container tray and placed at a predefined storage location.

In particular, in order to make the contents of the sterile goods container accessible and to optionally be able to remove them from the sterile goods container tray, it is advantageous if the sterile goods container lid is lifted from the sterile goods container tray by the handling apparatus.

It is further advantageous if a sieve basket accommodated in the sterile goods container is automatically removed with the handling system for sterile goods containers. In this way, no operator is required to remove the sieve basket from the sterile goods container, in particular from the sterile goods container tray comprised thereby. Such an automatic handling of sterile goods containers enables, in particular, a use in connection with highly contaminated sterile goods containers that, for example, are contaminated with highly infectious material.

For an optimal handling of the sieve basket, it is advantageous if a tool apparatus is brought into positive-locking or substantially positive-locking engagement with the two swivel bracket holding elements of the sieve basket on which a swivel bracket is held so as to be pivotable about a swivel bracket pivot axis. As explained, the sieve basket can thus be removed from the sterile goods container in a defined and secure manner when, in particular, two tool apparatuses are brought into engagement with the two swivel bracket holding elements of the two swivel brackets in the described manner. Furthermore, it also has advantages if a tool apparatus is brought into force-locking engagement with the two swivel bracket holding elements of the sieve basket on which a swivel bracket is held so as to be pivotable about a swivel bracket pivot axis. Thus, a sieve basket can be handled even if the tool apparatus is not specially configured, for example, to engage with the two swivel bracket holding elements in a positive-locking or substantially positive-locking manner. The tool apparatus can thus be used universally.

Advantageously, the sieve basket is lifted out of the sterile goods container with the handling apparatus. The sterile goods container can thus be completely emptied. In particular, two or more sieve baskets can thus be removed from a correspondingly large sterile goods container.

Furthermore, the use of one of the handling systems for sterile goods containers described above for performing one of the methods described above is proposed.

### BRIEF DESCRIPTION OF THE DRAWINGS

The subsequent description of preferred embodiments of the present disclosure serves in conjunction with the drawings for further explanation. In the drawings:

FIG. 1 shows a schematic perspective partial view of an embodiment of a handling system for sterile goods containers with a closed sterile goods container in the storage position;

FIG. 2 shows a view similar to FIG. 1 upon a tool apparatus of the handling system for sterile goods containers being brought into engagement with a locking bracket on the sterile goods container lid of the sterile goods container;

FIG. 3 shows an enlarged partial view of the region A in FIG. 2;

FIG. 4 shows a section view along line 4-4 in FIG. 3;

FIG. 5 shows a schematic perspective view similar to FIG. 3, with the locking bracket pivoted with the tool apparatus;

FIG. 6 shows a section view along line 6-6 in FIG. 5;

FIG. 7 shows a schematic view similar to FIG. 2, upon the sterile goods container lid being lifted from the sterile goods container tray with the handling system for sterile goods containers;

FIG. 8 shows an enlarged perspective partial view of the arrangement from FIG. 7;

FIG. 9 shows an enlarged perspective partial exploded depiction of an embodiment of a sterile goods container lid in the region of a locking mechanism;

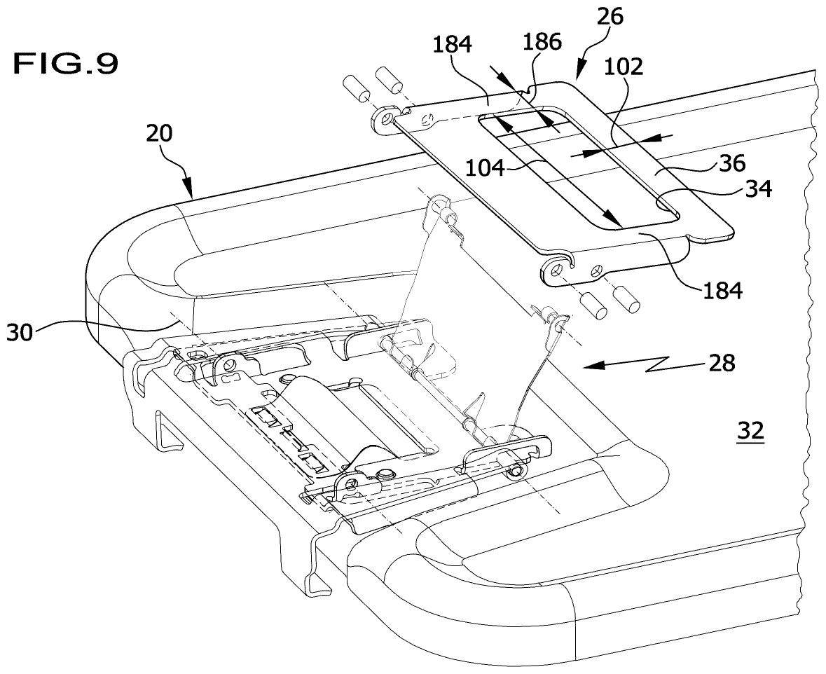

FIG. 10 shows a schematic, partial section view of an embodiment of an actuator device of the handling system for sterile goods containers;

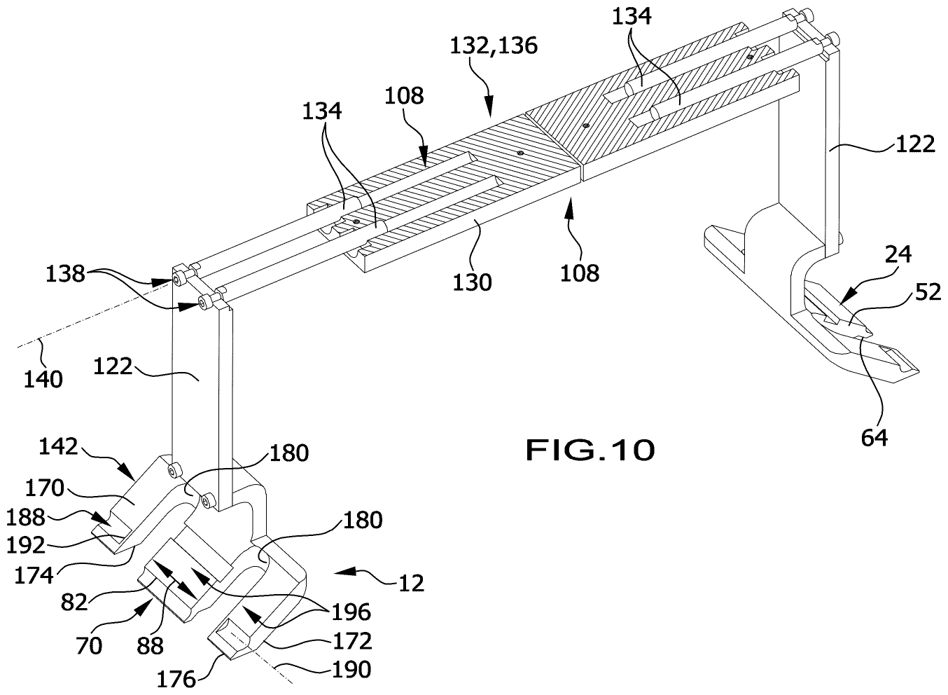

FIG. 11 shows a schematic perspective total view of an embodiment of a handling system for sterile goods containers upon removal of a sieve basket from a sterile goods container tray;

FIG. 12 shows an enlarged partial view of the region B from FIG. 11;

FIG. 13 shows a plan view of the arrangement from FIG. 12; and

FIG. 14 shows a section view along line 14-14 in FIG. 13.

### DETAILED DESCRIPTION

Schematically depicted in the Figures is a first embodiment of a handling system 10 for sterile goods containers. In particular, it is configured to automatically open sterile goods containers 14.

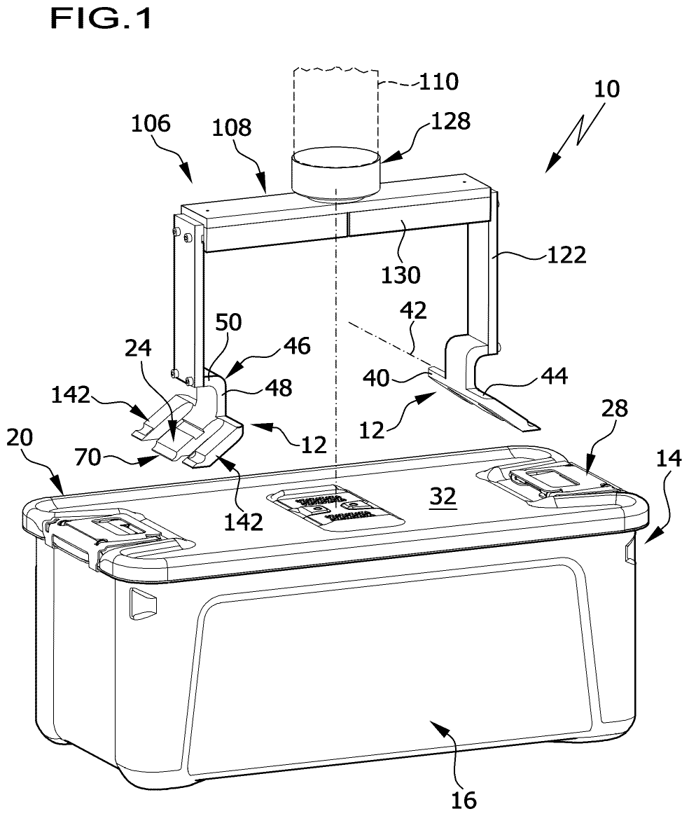

The sterile goods container 14 depicted schematically in FIG. 1 comprises a sterile goods container tray 16, which defines a receiving space 18, and a sterile goods container lid 20.

One or more sieve baskets 22 are able to be accommodated in the sterile goods container 14, depending on their size. In particular, medical instruments and/or implants, which are not shown in the Figures for the sake of clarity, are able to be accommodated in said sieve baskets, namely both for cleaning and sterilization purposes, and also for storage and transport purposes.

An essential element of the handling system 10 for sterile goods containers is a tool apparatus, which is denoted with the reference numeral 12. In the embodiment depicted in the Figures, the handling system 10 for sterile goods containers comprises two identically configured tool apparatuses 12. They are configured, in particular, to open the sterile goods container 14, to lift the sterile goods container lid 20, which is unlocked from the sterile goods container tray 16, and to lift out the sieve basket 22 accommodated in the sterile goods container tray 16 in the receiving space 18.

In the following, for the sake of simplicity, one of the two tool apparatuses 12 will be described in detail.

The tool apparatus 12 comprises a first tool element 24 for being brought into engagement with a locking bracket 26 of a container closure 28, which is denoted as a whole with the reference numeral 28. The structure of the container closure 28 is described in detail in DE 10 2018 117 046 A1, such that reference can be made to said published patent application in this regard.

The locking bracket 26, which is mounted on the sterile goods container lid 20 so as to be pivotable about a pivot axis 30, serves to actuate the container closure 28. The pivot axis 30 extends in parallel with a lid top side 32 of the sterile goods container lid 20.

In a storage position as it is schematically depicted in FIG. 1, the locking bracket 26 is oriented substantially in parallel with the lid top side 32 for locking the sterile goods container 20 on the sterile goods container tray 16 in a closing position, in which the sterile goods container lid 20 closes the sterile goods container tray 16.

In order to be able to lift the sterile goods container lid 20 from the sterile goods container tray 16, the container closure 28 must be unlocked. To this end, the locking bracket 26 must be pivoted from the storage position away from the lid top side 32 about the pivot axis 30.

In the embodiment of the sterile goods container 14 depicted in the Figures, the locking bracket 26 is of substantially plate-shaped configuration and comprises a substantially rectangular engagement portion 34, which extending away from the pivot axis 30 and in parallel thereto is delimited by a transverse web 36. The transverse web 36 forms a sort of free end of the locking bracket 26, which in the storage position points in the direction toward the other, identically configured container closure 28.

The tool apparatus 12 as a whole is configured mirror-symmetrically to a midplane 38. It comprises an elongate cuboidal support element 40, which defines a support element longitudinal axis 42. Formed projecting from a support element top side 44 is an L-shaped angle piece 46, with a first leg 48 projecting toward the top and a second leg 50 projecting perpendicularly therefrom toward the front.

The angle piece 46 is configured mirror-symmetrically to itself relative to the midplane 38.

The first tool element 24 projects from the support element 40 obliquely forward and downward and symmetrically to itself relative to the midplane 38. It has a tool element base body 52.

The tool element base body 52 has a base body top side 54 and a base body bottom side 56 pointing away from said top side. The base body top side 54 defines a base body sliding face 58. The base body bottom side 56 defines a base body bottom side face 60. The base body bottom side face 60 extends in parallel with the base body sliding face 58.

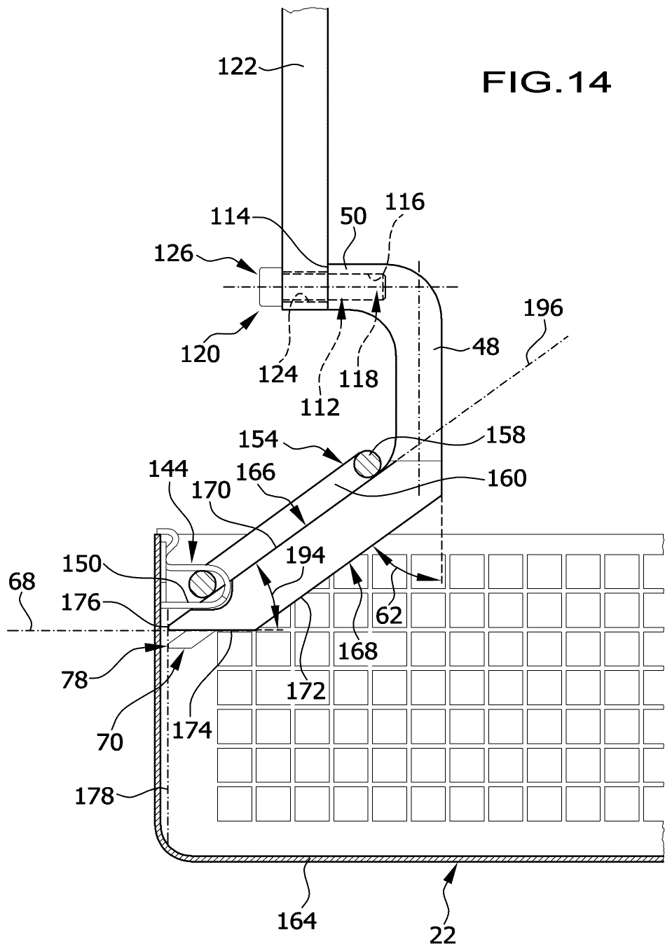

The base body bottom side face 60 together with the first leg 50 enclose a base body angle 62, which has a value in a range of about 30° to about 60°. In the embodiments depicted in the Figures, the base body angle 62 is about 55°, as schematically drawn in FIG. 14.

In addition to the base body sliding face 58, which serves for lifting and opening the locking bracket 26, the tool element base body 52 comprises a base body abutment face 64, which extends in parallel or substantially in parallel with the second leg 50.

The base body abutment face 64 and the base body sliding face 58 enclose a base body wedge angle 66. Said angle has a value in a range of about 10° to about 50°. In the embodiment depicted in the Figures, the base body wedge angle 66 is almost 40°.

The base body abutment face 64 defines an abutment face plane 68.

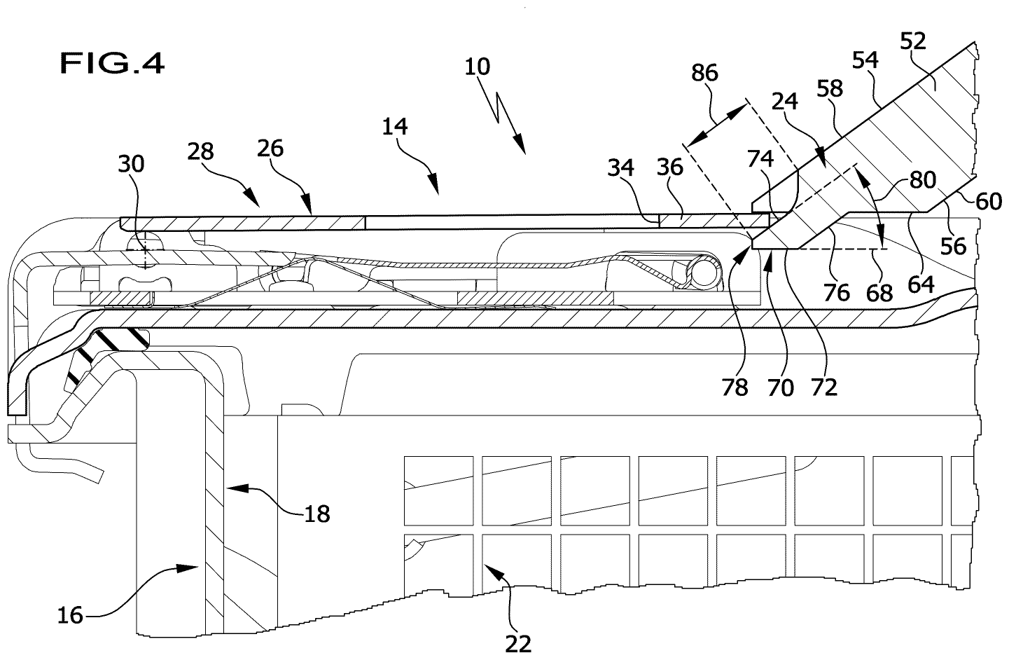

The first tool element 24 comprises a lifting wedge 70, which is configured to engage under the locking bracket 26 in the storage position, as can be seen schematically in FIG. 4. The lifting wedge 70 is arranged or formed distally, i.e., on a free end of the tool element base body 52.

The lifting wedge 70 has a lifting wedge abutment face 72 and a lifting wedge sliding face 74. The lifting wedge sliding face 72 extends in parallel with the base body abutment face 64.

As can be seen, for example, in FIG. 4, the lifting wedge 70 projects from the tool element base body 52 downward beyond the abutment face plane 68.

A rear side 76 of the lifting wedge 70 extends in parallel with the lifting wedge sliding face 74.

The lifting wedge abutment face 72 and the lifting wedge sliding face 74 intersect in a wedge edge 78, which is somewhat flattened off in the embodiment depicted in the Figures.

The lifting wedge abutment face 72, during the intended use of the tool apparatus 12, is oriented in parallel with the lid top side 32, as can be seen schematically in FIG. 4.

The lifting wedge abutment face 72 and the lifting wedge sliding face 74 enclose a wedge angle 80. Said angle has a value in a range of about 10° to about 50°. In the embodiment depicted in the Figures, the wedge angle 80 is almost 40°. In the embodiment depicted in the Figures, it corresponds to the base body wedge angle 66.

The rear side 76 extends in parallel with the base body bottom side face 60. The lifting wedge sliding face 74 extends in parallel with the base body sliding face 58, but is set back therefrom in the direction toward the base body bottom side face 60.

The lifting wedge sliding face 74 is of planar configuration commencing from the wedge edge 78, but then transitions in a concavely curved manner pointing away from the lifting wedge 70 into an orientation in parallel with the first leg 48, such that an edge 82 is formed in the transition between the lifting wedge sliding face 74 and the base body sliding face 58.

The lifting wedge sliding face 74 has a lifting wedge sliding face length 84 in parallel with the wedge edge 78. A lifting wedge sliding face width 86 perpendicular to the wedge edge 78 is drawn schematically in FIG. 4, namely in parallel with the base body sliding face 58. The lifting wedge sliding face length 84 is significantly greater than the lifting wedge sliding face width 86. In the embodiment depicted in the Figures, the lifting wedge sliding face length 84 is more than five times greater than the lifting wedge sliding face width 86.

The tool element base body 52 defines a base body width 88 in parallel with the wedge edge 78. The wedge edge 78 extends over the entire width of the tool element base body 52. Thus, the lifting wedge sliding face length 84 corresponds to the base body width 88.

A latching recess 90 that is recessed relative to the base body sliding face 58 is formed on the tool element base body 52 on the base body top side 56. It has a retaining face 92 pointing away from the lifting wedge 70 and a latching face 94 extending in parallel or substantially in parallel with the base body sliding face 58. The retaining face 92 extends transversely, namely perpendicularly in the embodiment depicted in the Figures, to the base body sliding face 58. Together with the base body sliding face 58, it forms a first intersection line and, with the latching face 94, forms a second intersection line 98.

The latching face 94 has a latching face width 100 perpendicular to the retaining face 92. In the case of the tool apparatus 12, the latching face width 100 is selected such that it is greater than a web width 102 of the transverse web 36 of the locking bracket 26 extending perpendicularly to the pivot axis 30. The transverse web 36 can thus be accommodated in the latching recess 90 over the entire web width 102.

The engagement portion 34 on the locking bracket 26 defines an engagement width 104 in parallel with the pivot axis 30. So that both the lifting wedge 70 and the tool element base body 52 are able to be inserted through the engagement portion 34, the tool element base body 52 is dimensioned in such a way that both the lifting wedge sliding face length 84 and the base body width 88 are smaller than the engagement portion width 104.

The transverse web 36 can dip into the latching recess 90 up to the latching face 94 and can contact the latching face 94, because the latching face 94 extends over the entire base body width 88.

The embodiment depicted in the Figures of the handling system 10 for sterile goods containers further comprises a handling apparatus 106 for moving, namely in particular for displacing, rotating, and/or pivoting, the tool apparatuses 12. The handling apparatus 106 comprises an actuator device 108, which is configured to move the tool apparatuses 12, in particular to displace, rotate, and/or to pivot the same.

The handling apparatus 106 further comprises a robot 110, which in FIG. 1 is only partially shown schematically with dashed lines. Said robot may be a robot 110 comprising a plurality of articulated arms, which are pivotable relative to one another and optionally are also rotatable about their longitudinal axes.

For directly or indirectly coupling to the mechanical handling apparatus 106 in a force-locking and/or positive-locking manner, the tool apparatus 12 comprises a first interface device 112. It comprises two first interface elements 118 in the form of blind holes 116. The two blind holes 116 provided with internal threads are arranged or formed on the angle piece 46, namely in face sides 114 of the angle piece 46.

The first interface elements 118 can be brought into engagement with corresponding second interface elements, for example the screws 120 depicted as an example in the Figures, in order to, for example, be screwed to a plate-shaped adapter element 122, which is provided with bores 124 corresponding to threaded portions of the screws 120. This is depicted schematically, in particular, in FIG. 14. A second interface element 126, which is associated with the handling apparatus 106, is thus formed by the screw 120 in connection with the bore 124.

The actuator device 108 comprises a second interface device 128 for directly or indirectly coupling to the robot 110 in a force-locking and/or positive-locking manner.

As described, in the embodiments depicted in the Figures of the handling systems 10 for sterile goods containers, the actuator device 108 is indirectly coupled or coupleable to two tool apparatuses 12 by way of the adapter element 122.

The actuator device 108 comprises a base body 130 on which a drive 132 is arranged. The drive 132 comprises four drive units 134, which are depicted purely schematically in the Figures. Here, they are linear drive components, so that the drive 132 in the embodiment depicted is configured in the form of a linear drive. In embodiments that are not depicted, the drive 132 comprises a linear drive and/or a pivot drive and/or a rotary drive.

The drive 132 may be configured, in particular, in the form of an electromotive drive and/or pneumatic drive 132.

The four drive units 134 are each coupled to the adapter element 122 by way of a further screw 138.

The described actuator device 108, in particular as it is schematically depicted in the Figures, enables a movement of the two tool apparatuses 12 toward one another or away from one another in a direction that runs parallel with a longitudinal axis 140 of the drive units 134.

The two tool apparatuses 12, as described, are indirectly coupled to the actuator device 108 by way of the adapter element 122, namely in such a way that the lifting wedges 70 point in opposite directions.

The actuator device 108 is optionally configured in such a way that two drive units that are connected to the same adapter element 122 are always able to be moved synchronously. This makes it possible, in particular, to move the two tool apparatuses 12 independently of one another relative to the base body 130. Optionally, they can of course be moved together, in particular synchronously, to the base body 130, namely selectively away from one another or toward one another or simultaneously in the one direction or the other.

The actuator device 108 is configured, in a manner that is not depicted in more detail, to move the tool apparatuses 12 in a path-controlled and/or force-controlled manner. For this purpose, in particular, force sensors may be provided on the drive units 134 or on the tool apparatuses 12.

Path-controlled means, in particular, that when a position and an orientation of the sterile goods container 14 in space is known and accordingly the position and orientation of the actuator device 108 in space is known, the drive 132 is controllable in such a way that the tool apparatuses 12 are able to each be brought into a certain position in the desired manner, in particular for being brought into engagement with the locking brackets 26 on the sterile goods container lid 20.

The tool apparatuses 12 as a whole are of symmetrical configuration relative to the midplane 38 extending perpendicularly to the wedge edge 78. Furthermore, the first tool element 24 is arranged or formed projecting transversely relative to the support element longitudinal axis 42 defined by the support element 40.

The two tool apparatuses 12 furthermore each comprise two second tool elements 142. These serve to be brought into positive-locking or substantially positive-locking engagement with swivel bracket holding elements 144 arranged or formed on the sieve basket 22. On each of the face sides 146 extending in parallel with one another, pointing into an inner space 148 of the sieve basket 22, two respective swivel bracket holding elements 144 are arranged in pairs. They define substantially elongate hole-like receptacles 150 for free end portions 152 of a swivel bracket 154 that point toward one another. The end portions 152 pointing toward one another define a swivel bracket pivot axis 156, about which the swivel bracket 154 is pivotable.

The swivel bracket 154 comprises a handle portion 158 extending rectilinearly and in parallel with the face sides 146 and comprises two connecting portions 160 arranged mirror-symmetrically, which connect the end portions 152 and the handle portion 158.

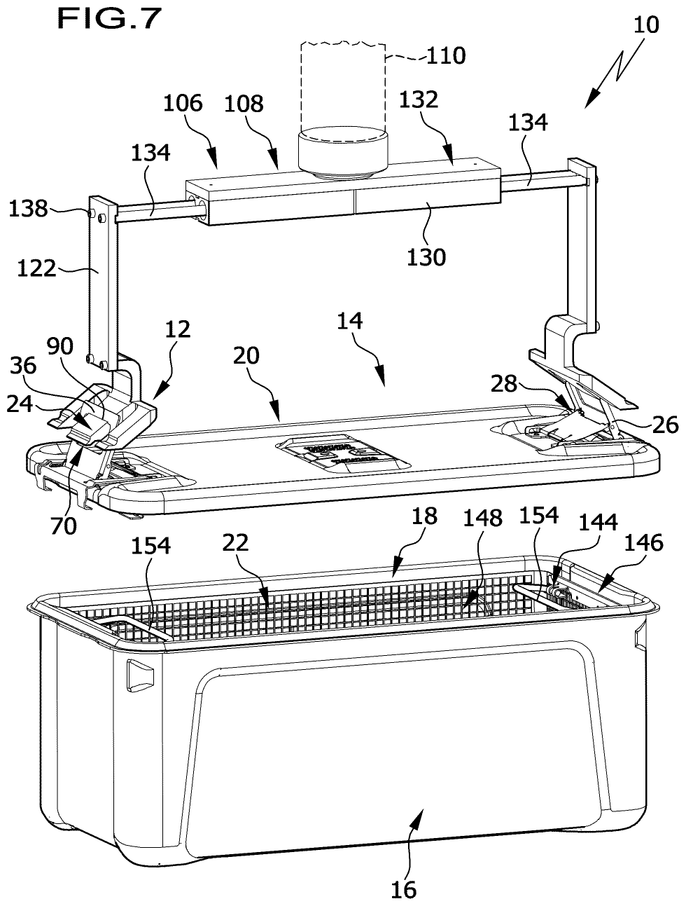

In a storage position as is schematically depicted in FIG. 7, the swivel brackets 154 are supported on lateral supports, which are formed on each face side 146 on one of the swivel bracket holding elements 144, such that the regions of the swivel bracket 154 defining a common plane, namely the handle portion 158 and the two connecting portions 160, are oriented in parallel or substantially in parallel with a base 164 of the sieve basket 22.

The two second tool elements 142 are also arranged or formed on the support element 40 and extend substantially in parallel with the first tool element 24 away from the support element 40.

Each second tool element 142 comprises a tool element top side 166 and a tool element bottom side 168 pointing away from said tool element top side 166. The tool element top side 166 defines a tool element sliding face 170. The tool element bottom side 168 defines a tool element bottom side bottom face 172 extending in parallel with the tool element sliding face 170.

The second tool element 142 further has a tool element abutment face 174. It forms part of the tool element bottom side 168. It is inclined against the tool element bottom side face 172 and encloses with it an obtuse angle, as is schematically depicted in FIG. 14.

The tool element abutment face 174 extends in parallel or substantially in parallel with the base body abutment face 64 and with the lifting wedge abutment face 72. In the embodiment depicted in the Figures, the tool element abutment face 174 lies in the abutment face plane 68.

The tool element abutment face 174 and the tool element sliding face 170 intersect in a front edge 176 of the second tool element 142. The front edge 176 and the wedge edge 78 together define a plane 178, which extends in parallel with the first leg 48.

In the embodiment depicted, the tool apparatuses 12 comprise two second tool elements 142. They are arranged or formed mirror-symmetrically to the midplane 38.

A respective cutout 180 is formed between the first tool element 24 and the two second tool elements 142. A cutout width 182 corresponds to a distance between the tool elements 24 and 142. Thus, the first tool element 24 is arranged or formed between the two second tool elements 142.

The locking bracket 26 comprises two legs 184, which extend from the transverse web 36 in parallel with one another and have a leg width 186 in a direction parallel with the transverse web 36. The cutout width 182 is selected such that the legs 184 are each able to engage into one of the two cutouts 180. The leg width 186 is thus smaller than the cutout width 182.

The cutouts 180 each extend from free ends of the tool elements 24 and 142 up to the support element 40.

For being brought into positive-locking engagement with the swivel bracket holding elements 144, a coupling receptacle 188 is formed on each of the two second tool elements 142 of each tool apparatus 12. They have an inner contour that is adapted to an outer contour of the swivel bracket holding elements 144, such that they are able to dip into the coupling receptacles 188 when the tool apparatus 12 is oriented and moved in parallel with the base 164 of the sieve basket 22.

The coupling receptacles 144 are each arranged or formed on the tool element top side 166.

The coupling receptacles 188 of the two second tool elements 142 of the same tool apparatus 12 define a common coupling receptacle longitudinal axis 190. They extend in parallel or substantially in parallel with the swivel bracket pivot axis 156 when a swivel bracket holding element 144 is accommodated in each coupling receptacle 188 of the two second tool elements 142.

A transverse stop 192 is arranged or formed on each coupling receptacle 188 for delimiting a movement of the tool apparatus 12 relative to a sterile goods container 14 or to the sieve basket 22 in a direction parallel with the swivel bracket pivot axis 156 when the swivel bracket holding elements 144 are accommodated in the coupling receptacles 188. The transverse stops 192 delimit the coupling receptacles 188 laterally in each case in the direction toward the first tool element 24.

The tool element sliding face 170 of the second tool element 142 serves to lift the swivel bracket 154 of the sieve basket 22. The tool element abutment face 174 and the tool element sliding face 170 enclose a tool element wedge angle 194. In the embodiment depicted in the Figures, the tool element wedge angle 194 corresponds to the base body wedge angle 66.

Furthermore, the tool element sliding face 170 and the base body sliding face 58 define a common sliding face plane 196. The tool element bottom side face 172 and the base body bottom side face 60 also lie in a common plane.

In the embodiment depicted in the Figures, the tool apparatuses 12 are formed in one piece, namely monolithically.

They are made of a metallic material, namely by being machined from a solid material block. In the embodiment described, the metallic material is an instrument steel that is sterilizable by superheated steam. However, other metallic materials that are corrosion-resistant are also suitable for forming the tool apparatuses 12.

In particular, a method for machine handling of sterile goods containers 14 can be performed with the described handling system 10 for sterile goods containers. Here, in particular, it may be a sterile goods container 14 as described above, which has a sterile goods container lid 20 and a sterile goods container tray 16.

When a sterile goods container 14 is provided in the closing position, using the handling system 10 for sterile goods containers, the sterile goods container 14 can first be transferred from the closing position described above into an unlocking position. To achieve this, the tool apparatuses 12 are each brought into engagement with one of the two locking brackets 26 for pivoting the same from the closing position, in which the locking brackets 26 are oriented in parallel or substantially in parallel with the lid top side 32, about the pivot axis 32 into an unlocking position, in which the sterile goods container lid 20 and the sterile goods container tray 16 are unlocked.

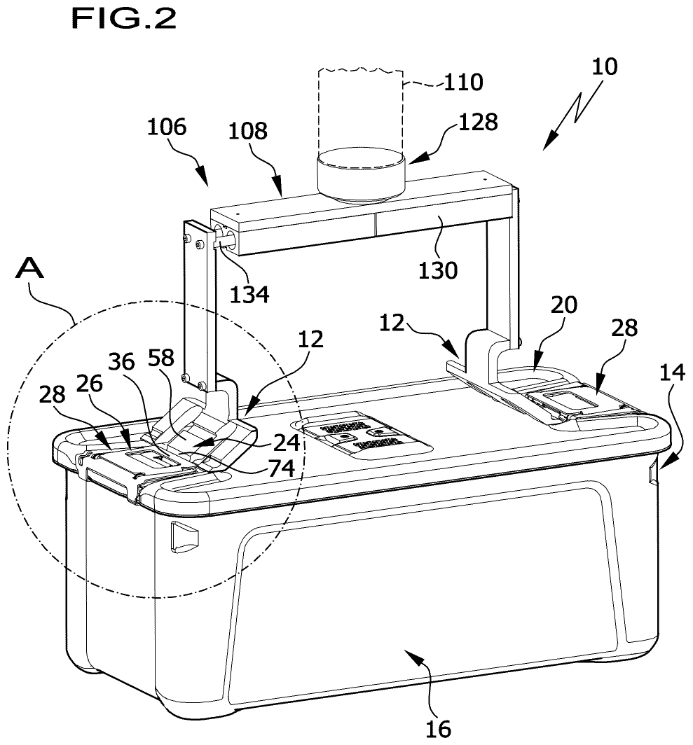

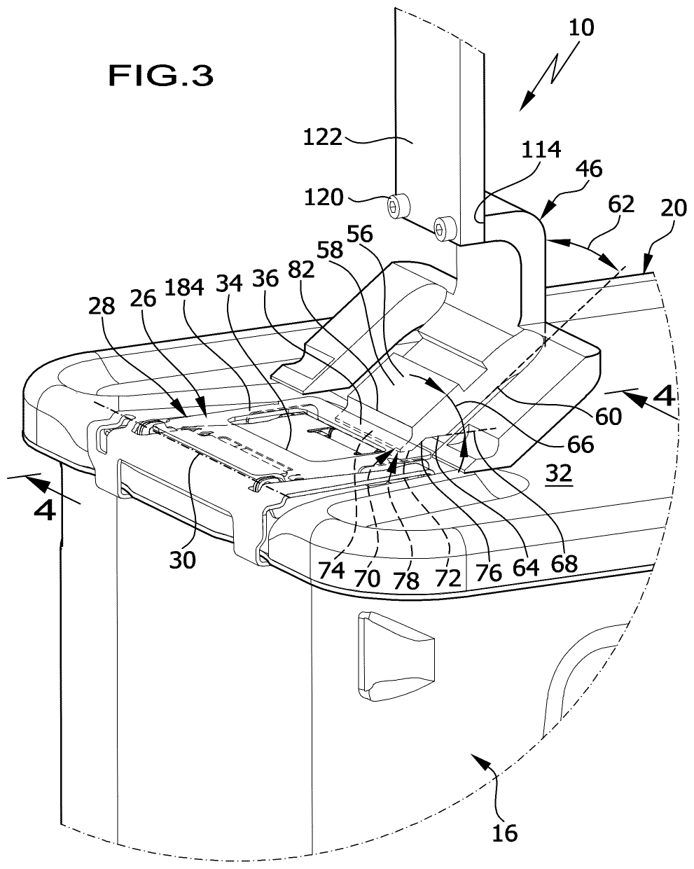

FIG. 1 shows the sterile goods container 14 in the closing position. The two tool apparatuses 12 are brought up to the locking brackets 26 by the handling apparatus 106 in such a way, either simultaneously or one after the other, that the lifting wedges 70 engage under the transverse webs 36 of the locking brackets 26, as is schematically depicted in FIGS. 2 to 4.

When the tool apparatuses 12 that with their lifting wedges 70 are in contact with the transverse webs 36 are displaced in parallel with the lid top side 32 in the direction toward the pivot axis 30, the transverse web 36 first slides on the lifting wedge sliding face 74 and when advanced further is moved over the edge 82 onto the base body sliding face 58. When the tool apparatus 12 is advanced further such that the transverse web 36 passes the first intersection line 96, the transverse web 36 snaps into the latching recess 90 and comes to rest on the latching face 94.

When the tool apparatus 12 is now moved back somewhat in the direction toward the other tool apparatus 12, the transverse web 36 is tensioned against the retaining face 92.

In the described manner, the sterile goods container lid 20 is held and secured in a clamping manner on the two tool apparatuses 12.

The sterile goods container lid 20 can now be lifted from the sterile goods container tray 16 by the handling apparatus 106 and is placed at a desired storage location.

To release the sterile goods container lid 20 from the two tool apparatuses 12, they are moved relative to the sterile goods container lid 20 in the reverse direction as when being brought into engagement as described above.

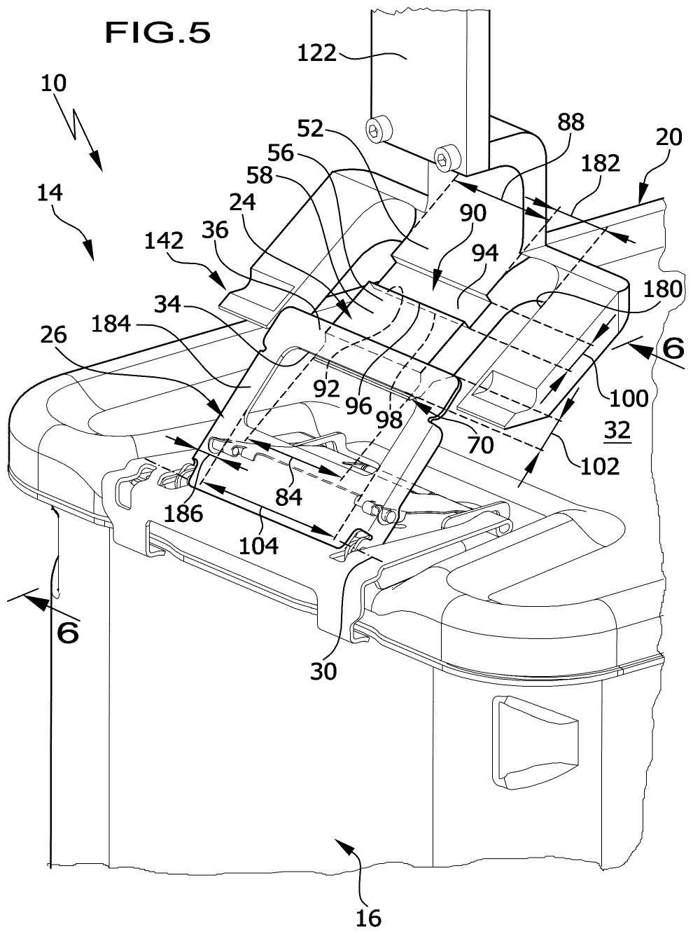

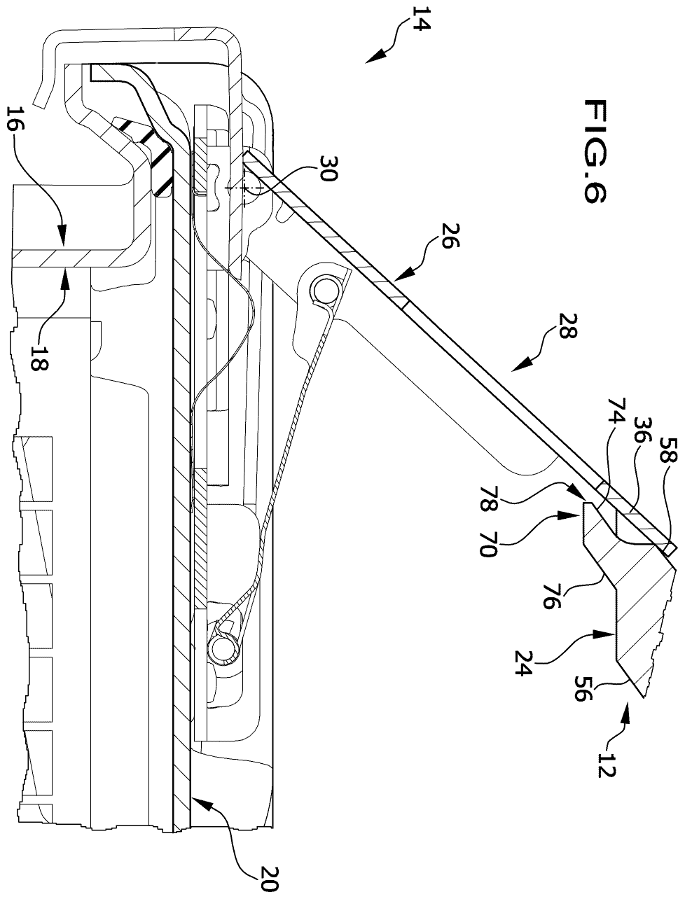

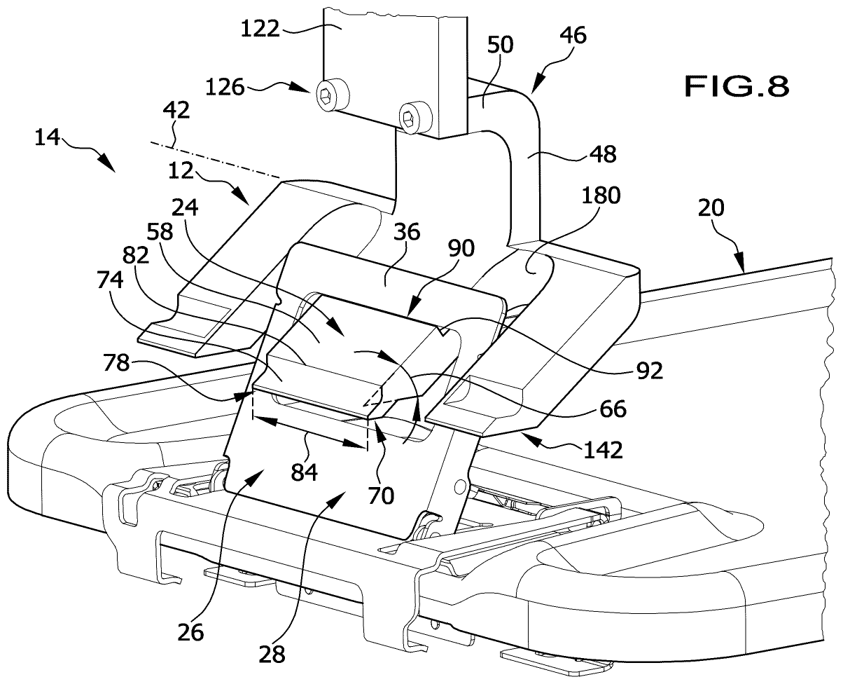

The sliding of the transverse web 36 on the first tool elements 24 is depicted in FIGS. 5 and 6. FIGS. 7 and 8 show the engagement of the transverse web 36 into the latching recess 90 on the first tool element 24. FIG. 7 shows the sterile goods container lid 20 being lifted from the sterile goods container tray 16.

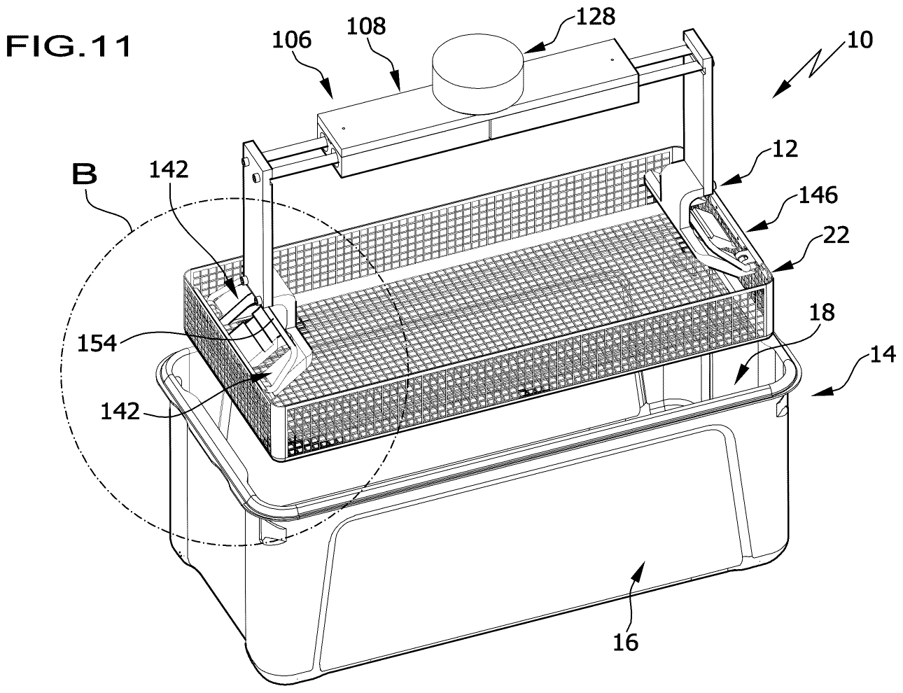

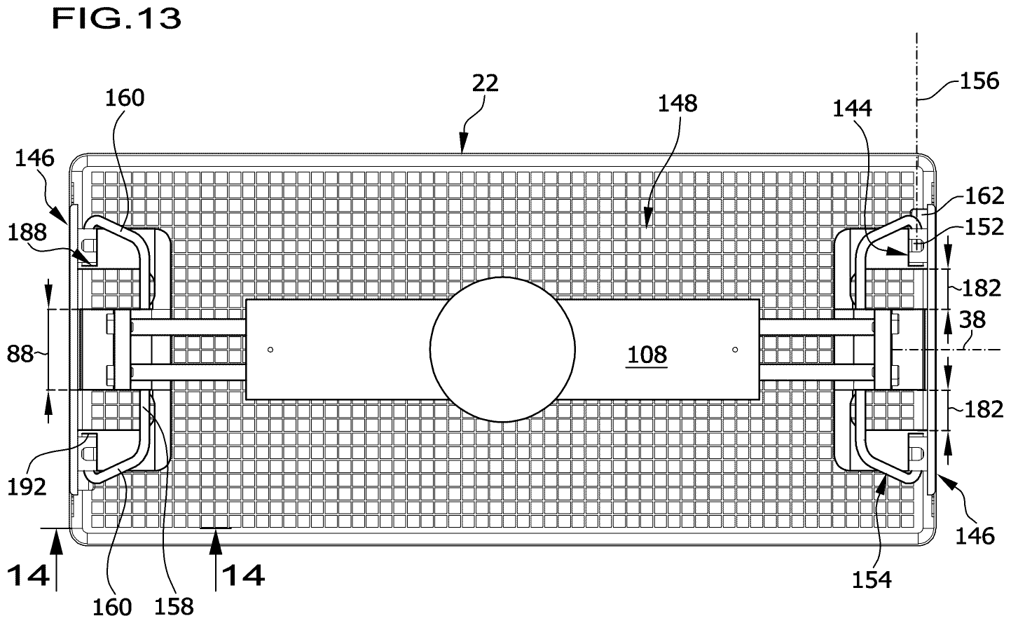

The sterile goods container 14 that is now open provides access to the sieve basket 22. In order to lift said sieve basket 22 out of the receiving space 18 using the handling apparatus 106, the two tool apparatuses 12, as described and depicted as an example in FIGS. 11 to 14, are brought with the tool element sliding faces 170 into contact with the handle portions 158. When the tool apparatuses 12 are then moved in parallel with the base 164 in the direction toward the swivel bracket holding elements 144, the swivel bracket holding elements 144 are able to dip into the coupling receptacles 188 provided therefor. The four total swivel bracket holding elements 144 are held in said coupling receptacles 188 by the weight of the sieve basket 22.

The sieve basket 22 is secured in a plane parallel to the base 164 against a movement relative to the handling apparatus 106, on the one hand by the configuration of the coupling receptacles 188, which each point in pairs in different directions, namely toward the two face sides 146 of the sieve basket 22, and on the other hand by the transverse stops 192, which prevent a movement of the sieve basket 22 relative to the handling apparatus 106 in parallel with the swivel bracket pivot axis 156.

When the tool apparatuses 12 are moved in the direction toward the face sides 146 of the sieve basket 22, the handle portions 158 slide on the tool element sliding faces 170. It is not possible for the handle portion 158 to dip into the latching receptacle 90, because the handle portion 158 has a length such that it is always supported on the two tool element sliding faces 170 on both sides of the first tool element 24 arranged between the second tool elements 142.

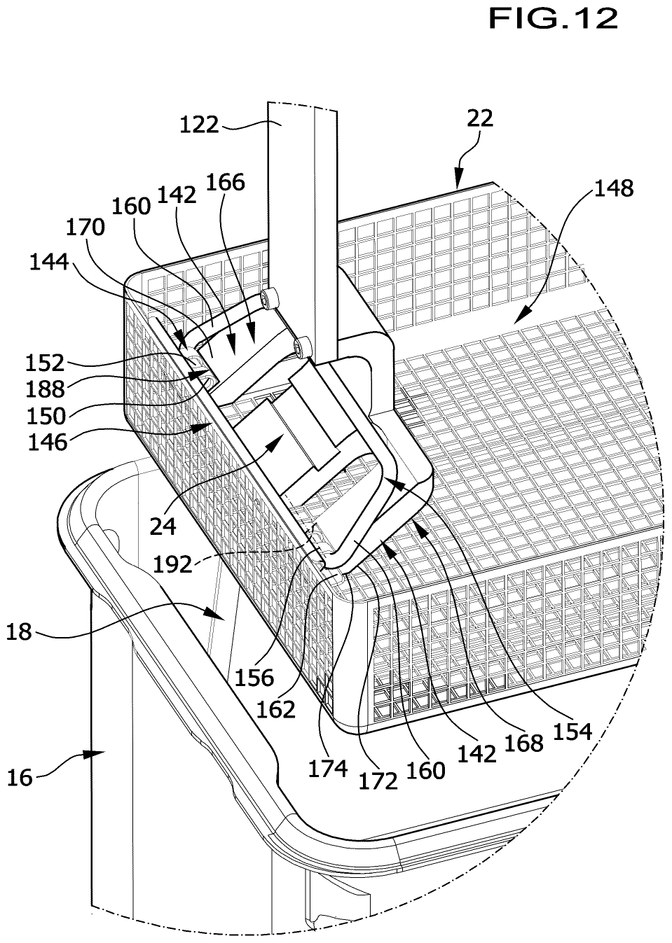

The sieve basket 22 coupled to the tool apparatuses 12 as described can now be lifted out of the sterile goods container tray 16, as depicted schematically in FIGS. 11 and 12. In a next step, it can be placed at a location where its contents can be removed by a user, either by hand or by machine with a further handling apparatus.

The handling system 10 for sterile goods containers makes it possible, in particular, to fully automatically handle sterile goods containers 14 and also sieve baskets 22 accommodated by said containers.

The sterile goods containers 14 and the sieve baskets 22 may optionally be optically and/or electronically marked so that the correspondingly configured handling system 10 for sterile goods containers is able to automatically detect their size and position and/or orientation in space and thus is able to automatically handle all components of the sterile goods container 14 accordingly.

The described handling system 10 for sterile goods containers simplifies and facilitates a handling of sterile goods containers 14, in particular for personnel in reprocessing facilities, as are provided, e.g., in hospitals or corresponding companies that are specialized in the cleaning and sterilization of sterile goods containers 14 and their contents.

### REFERENCE NUMERAL LIST

10 handling system for sterile goods containers
12 tool apparatus
14 sterile goods container
16 sterile goods container tray
18 receiving space
20 sterile goods container lid
22 sieve basket
24 first tool element
26 locking bracket
28 container closure
30 pivot axis
32 lid top side
34 engagement portion
36 transverse web
38 midplane
40 support element
42 support element longitudinal axis
44 support element top side
46 angle piece
48 first leg
50 second leg
52 tool element base body
54 base body top side
56 base body bottom side
58 base body sliding face
60 base body bottom side face
62 base body angle
64 base body abutment face
66 base body wedge angle
68 abutment face plane
70 lifting wedge
72 lifting wedge abutment face
74 lifting wedge sliding face
76 rear side
78 wedge edge
80 wedge angle
82 edge
84 lifting wedge sliding face length
86 lifting wedge sliding face width
88 base body width
90 latching recess
92 retaining face
94 latching face
96 first intersection line
98 second intersection line
100 latching face width
102 web width
104 engagement portion width
106 handling apparatus
108 actuator device
110 robot
112 first interface device
114 face side
116 blind hole
118 first interface element
120 screw
122 adapter element
124 bore
126 second interface element
128 second interface device
130 base body
132 drive
134 drive unit
136 linear drive
138 screw
140 longitudinal axis
142 second tool element
144 swivel bracket holding element
146 face side
148 inner space
150 receptacle
152 end portion
154 swivel bracket
156 swivel bracket pivot axis
158 handle portion
160 connecting portion
162 support
164 base
166 tool element top side
168 tool element bottom side
170 tool sliding face
172 tool element bottom side face
174 tool element abutment face
176 front edge
178 plane
180 cutout
182 cutout width
184 leg
186 leg width
188 coupling receptacle
190 coupling receptacle longitudinal axis
192 transverse stop
194 tool element wedge angle
196 sliding face plane

1. A handling system for sterile goods containers, the handling system comprising:
at least one tool apparatus for opening sterile goods containers,
the at least one tool apparatus having at least one first tool element comprising a lifting wedge,
the at least one first tool element being engageable with a locking bracket mounted on a sterile goods container lid of a sterile goods container so as to be pivotable about a pivot axis,
the at least one first tool element being configurable in a storage position to be oriented parallel to or substantially parallel to a lid top side of the sterile goods container lid for locking the sterile goods container lid on a sterile goods container tray of the sterile goods container in a closing position in which the sterile goods container lid closes the sterile goods container tray, and
the lifting wedge being configured for engaging under the locking bracket.

2. The handling system according to claim 1, wherein:
the lifting wedge has a lifting wedge abutment face and a lifting wedge sliding face,
the lifting wedge abutment face is configured to be oriented in parallel with the lid top side, and
the lifting wedge abutment face and the lifting wedge sliding face enclose a wedge angle.

3. The handling system according to claim 2, wherein the lifting wedge sliding face is of planar or curved configuration.

4. The handling system according to claim 2, wherein the wedge angle has a value in a range of about 10° to about 50°.

5. The handling system according to claim 2, wherein:
the lifting wedge abutment face and the lifting wedge sliding face intersect at a wedge edge,
the lifting wedge sliding face has a lifting wedge sliding face length parallel to the wedge edge and a lifting wedge sliding face width perpendicular to the wedge edge, and
the lifting wedge sliding face length is greater than the lifting wedge sliding face width.

6. The handling system according to claim 5, wherein the at least one tool apparatus is of symmetrical configuration relative to a midplane extending perpendicularly to the wedge edge.

7. The handling system according to claim 5, wherein:
the at least one first tool element has a tool element base body, and
the lifting wedge is arranged or formed distally on the tool element base body.

8. The handling system according to claim 7, wherein:
the tool element base body has a base body abutment face and a base body sliding face for lifting and opening the locking bracket, and
the base body abutment face and the base body sliding face enclose a base body wedge angle.

9. The handling system according to claim 8, wherein:
the tool element base body has a base body top side that defines the base body sliding face, and
the base body top side defines a latching recess that is recessed relative to the base body sliding face.

10. The handling system according to claim 7, wherein:
the locking bracket has an engagement portion,
the engagement portion defines an engagement portion width in parallel with the pivot axis,
the lifting wedge sliding face has a lifting wedge sliding face length,
the tool element base body has a base body width parallel to the wedge edge, and
the lifting wedge sliding face length and the base body width are smaller than the engagement portion width.

11. The handling system according to claim 1, wherein the at least one tool apparatus comprises a first interface device for directly or indirectly coupling to a mechanical handling apparatus in a force-locking and/or positive-locking manner for moving the at least one tool apparatus.

12. The handling system according to claim 11, wherein:
the mechanical handling apparatus comprises an actuator device, and
the at least one tool apparatus comprises two tool apparatuses that are directly or indirectly held on the actuator device with lifting wedges pointing away from one another or toward one another.

13. The handling system according to claim 1, wherein the at least one tool apparatus comprises at least one second tool element configured to be brought into positive-locking engagement with a swivel bracket of a sieve basket that is insertable into a sterile goods container and/or with a swivel bracket holding element holding the swivel bracket on the sieve basket so as to be pivotable about a swivel bracket pivot axis, the at least one first tool element and the at least one second tool element being separated by a cutout.

14. The handling system according to claim 13, wherein the at least one second tool element comprises a coupling receptacle configured to be brought into positive-locking engagement with the swivel bracket holding element of the sieve basket.

15. The handling system according to claim 14, wherein the at least one second tool element comprises two second tool elements.

16. The handling system according to claim 15, wherein a transverse stop is arranged or formed on each coupling receptacle for delimiting a movement of the at least one tool apparatus relative to the sterile goods container in a direction parallel with the swivel bracket pivot axis when swivel bracket holding elements are accommodated in the coupling receptacles.

17. The handling system according to claim 1, wherein the at least one tool apparatus is formed in a one-piece monolithic body of unitary construction.

18. A method for machine handling of sterile goods containers, the sterile goods containers each having a sterile goods container tray and a sterile goods container lid, each sterile goods container lid supporting two locking brackets that are pivotable about a pivot axis for locking the sterile goods container lid on the sterile goods container tray in a closing position, the method comprising the steps of:
utilizing the handling system according to claim 1;
moving the at least one tool apparatus into engagement with a locking bracket of one of the sterile goods containers; and
pivoting the locking bracket from the closing position, in which the locking bracket is oriented in parallel or substantially in parallel with a lid top side of the sterile goods container lid, about the pivot axis into an unlocking position, in which the sterile goods container lid and the sterile goods container tray are unlocked.

19. The method according to claim 18, wherein:
a) a transverse web of the locking bracket is accommodated in a latching recess on the at least one first tool element and held under tension against a retaining face,
and/or
b) a sieve basket accommodated in the sterile goods container is automatically removed with the handling system.

20. A handling system for sterile goods containers, the handling system comprising:
at least one tool apparatus for opening sterile goods containers,
the at least one tool apparatus having at least one first tool element comprising a lifting wedge,
the at least one first tool element being engageable with a locking bracket mounted on a sterile goods container lid of a sterile goods container so as to be pivotable about a pivot axis,
the at least one first tool element being configurable in a storage position to be oriented parallel to or substantially parallel to a lid top side of the sterile goods container lid for locking the sterile goods container lid on a sterile goods container tray of the sterile goods container in a closing position in which the sterile goods container lid closes the sterile goods container tray,
the lifting wedge being configured for engaging under the locking bracket, and
the at least one tool apparatus having at least one second tool element configured to be brought into positive-locking engagement with a swivel bracket of a sieve basket that is insertable into a sterile goods container and/or with a swivel bracket holding element holding the swivel bracket on the sieve basket so as to be pivotable about a swivel bracket pivot axis.
